(19) United States
(12) Patent Application Publication
(10) Pub. No.: US 2010/0137774 A1
(43) Pub. Date: Jun. 3, 2010

(54) MEDICAL COMPRESS I

(75) Inventors: Juergen Hofstetter, Heidenheim (DE); Horst Schmid, Sontheim (DE); Rainer Halbauer, Obergroeningen (DE)

Correspondence Address: Brinks Hofer Gilson & Lione/Ann Arbor, 524 South Main Street, Suite 200, Ann Arbor, MI, 48104, US

(73) Assignee: Paul Hartmann AG, Heidenheim (DE)

(21) Appl. No.: 12/697,395

(22) Filed: Feb. 1, 2010

Related U.S. Application Data
(63) Continuation of application No. PCT/EP2008/005718, filed on Jul. 12, 2008.

(30) Foreign Application Priority Data
Aug. 1, 2007 (DE) 10 2007 036 083.7

(51) Int. Cl. A61F 13/00; B23P 17/04

(52) U.S. Cl. 602/44; 29/428

(57) ABSTRACT

The invention relates to medical compresses, especially gauze compresses that can be especially advantageously folded. The invention also relates to a stack comprising a plurality of compresses, and a method for producing said compresses.

### CROSS-REFERENCE TO RELATED APPLICATIONS

This application is a continuation of International Application No. PCT/EP2008/005718 filed on Jul. 12, 2008, which claims the benefit of DE 10 2007 036 083.7, filed Aug. 1, 2007. The disclosures of the above applications are incorporated herein by reference.

### FIELD

The present disclosure relates to medical compresses, in particular gauze compresses, in a user-friendly form. The disclosure also relates to a method of producing such compresses.

### BACKGROUND

The statements in this section merely provide background information related to the present disclosure and may not constitute prior art.

Medical compresses for treating acute wounds in emergency medicine or for use during surgical procedures have been known for some time. These compresses are substantially distinguished from each other with reference to the materials used, and on the basis of these distinctions, are divided into gauze compresses and non-woven fabric compresses. Gauze compresses are normally manufactured from cotton fabric, which, depending on the yarn density, has a coarse or a fine lattice structure. The requirements for gauze used in compresses are specified by DIN EN 14079.

Because gauze compresses involve a lattice structure, which has the disadvantage that fibers at the ends of the compress can become detached, a plurality of solutions have been presented to prevent such detachment. In DE 2261889, for example, a compress is described that features at least one intermeshed strip. The intermeshing of the threads forms two opposite edges of a tape material from which no threads can become detached. The compresses are ultimately formed from a partial section of this tape material, wherein the cut edges of this section are folded in. Furthermore, DE 9014500 discloses a gauze compress that has thermoplastic threads, bands, strips, or non-woven strips in the immediate vicinity of a cut edge. These additional materials are fused, sealed with the gauze, or glued to it. ES compresses are an example of known gauze compresses that have become established on the market. What the proposed solutions and existing products all have is common is that the production thereof is considered too complex and/or too cost-intensive.

### SUMMARY

The present disclosure provides textile compresses that can be used securely and the production of which is cost-efficient. In addition, it should be easy to stack a plurality of such compresses, the compresses requiring as little packaging volume as possible. In addition, a method for manufacturing medical compresses is being provided.

This is achieved by a medical compress according to claim 1. According to this claim, a medical compress according to the present disclosure comprises at least 8 layers of a flat web textile material, wherein each layer is connected via a folded edge to an additional layer, and wherein at least one first folded edge and a second folded edge are situated perpendicular to each other. The compress is folded in such a way that the compress comprises at least two folded edges as first hems, wherein each of these first hems connects directly adjacent layers or partial sections of the directly adjacent layers. In particular, the medical compress comprises two folded edges as first hems and two cut edges as second hems.

In the context of the present disclosure, a folded edge shall be understood as an edge or a partial section of the edge that is formed by the complete or partial superposition, laying, or folding of two subunits of the material section over each other, the two subunits of the material section being connected via the folded edge. The two subunits of the section of material can be directly adjacent after being superimposed, laid, or folded over each other that is the two subunits of the section of material can be in direct contact or separated by additional layers.

By comparison, a cut edge of a material section of the flat web material is an edge that is formed by severing a first material section from larger material section comprising the first material section, wherein a cut edge can be associated with the first material section and a cut edge with the remaining material section, respectively. Here, all of the severing techniques known today, such as cutting with a knife or scissors, laser beams, water jets, or other techniques can be employed.

In addition, a hem shall be understood as a cut or folded edge, as well as a partial section of said cut or folded edge that is formed by placing said cut or folded edge, or said partial section of the cut or folded edge, against an additional cut edge or an additional folded edge, or against partial sections of the additional cut edge or the additional folded edge, wherein the cut or folded edges involved, or the partis sections thereof, are located in one plane. Ideally, the hems of a compress, according to the disclosure, rest directly adjacent from or against each other. According to the present disclosure, however, edges will also be referred to as hems if the cut or folded edges involved are located at a negligible distance from each other, that is no more than about 15%, and/or overlap by no more than about 15%, wherein the respective value of the distance or the overlap refers to the length of a folded edge of the compress in the finished folded state, and the folded edge of the compress that has the greatest value in numerical terms is used as a basis for measurement.

Furthermore, in the context of the present disclosure (unless otherwise indicated), a folded edge is understood as an outer edge of the finished compress.

By forming at least two folded edges as a first hem, a compress can be produced which has no exposed cut edges and which will also save material and thus permit a cost reduction during manufacture. The formation of hems also prevents threads from becoming detached from the cut edge and getting into a wound when the compress is used as intended. In addition, a compress according to the present disclosure has the advantage that even when the finished compress, in particular the compress that has at least 8 layers, is folded open once, no cut edges are exposed. This compress can be used particularly securely, and what is more, it is user-friendly, because the user can decide whether he wants to use, for example, an 8-layer compress as a 4-layer or an 8-layer compress. No cut edges are exposed in either case.

It has also proven advantageous to have the compress folded in such a way that the outer layers of the compress forming the contact surfaces have the shape of a rectangle or a square. In particular, the medical compress is folded in such a way that the outer layers forming the contact surfaces of the compress are completely formed by a continuous region of the flat web material, which is to say have no hems, wherein the contact surfaces are still preferably rectangular or square. It is particularly preferred that the compress be folded in such a way that all outer edges of the finished compress are formed by folded edges.

These contact surfaces can be obtained by manufacturing a compress according to the present disclosure from a rectangular section of flat web material that has two opposite, parallel cut edges A and two opposite parallel cut edges B, wherein the edge length a of the cut edges A is greater than or equal to the edge length b of the cut edges B, and each cut edge, folded edge, hem and/or outer edge of the compress is located parallel or perpendicular to an additional cut edge, folded edge, hem and/or outer edge of the compress, and wherein at least one cut edge, folded edge, or hem of the compress is located perpendicular to an additional cut edge, folded edge, or hem of the compress. In particular, the edge length a of the cut edges A is greater than the edge length b of the cut edges B. Furthermore, folded edges that are formed parallel to the cut edges B of the material section preferably form the first hems, wherein is particularly preferred for the cut edges A to form the second hems. In this way, a compress according to the present disclosure is formed, in particular from a rectangular material section with the cut edges A and B of the flat web textile material, wherein the edge length a of the cut edges A is greater than the edge length b of the cut edges B. In this way, a compress according to the present disclosure preferably also comprises rectangular or square contact surfaces with the outer edges C and D, wherein the edge length c of the outer edges C is greater than or equal to the edge length d of the outer edges D and is preferably folded in such a way that the first hems are located parallel to the first folded edges, and in particular parallel to the outer edges D. In particular, a compress according to the present disclosure has square contact surfaces F with outer edges D having the edge length d. However, it can also be provided that a compress according to the present disclosure comprises square contact surfaces with the outer edges C and D, wherein the edge length c of the outer edges C is greater than the edge length d of the outer edge D and comprises first folded edges located parallel to the outer edge D as the first hems. In this case, the cut edges A of the rectangular material section are located parallel to the outer edges C, and the cut edges B of the rectangular material section are located parallel to the outer edges D. It should be noted at this juncture that the compresses according to the present disclosure can be produced both by machine and by hand.

Fundamentally, a compress can be produced from a rectangular section of a flat web material with two parallel cut edges A that are located opposite of each other, and two parallel cut edges B that are located opposite of each other, wherein the edge length (a) of the cut edges A is greater than the edge length (b) of the cut edges B. However, when the first hems are formed by folded edges formed parallel to the cut edges B, in particular hems that are formed from first folded edges, in contrast to hems that are formed by folded edges parallel to the cut edges A, which is to say hems that are formed by two folded edges, considerable savings in material can be achieved. The material savings, for example, for a square, 8-layer compress made of the same material, amount to about 5-15%, depending on the contact surface and the width of the partial regions that are folded over first.

According to further forms, a compress according to the present disclosure is comprised of at least 8 and no more than 16 layers. In particular, a compress according to the present disclosure is a compress which, viewed in a cross-section, comprises 8-layer and 10- or 12-layer regions. This means that the compress, viewed in a cross-section, does not have a homogenous layer structure across the entire region of the transverse or longitudinal extension thereof, but rather that the compress has 10 or 12 layers in a first partial region, and that it has 8 layers in at least one additional partial region. In another form, the compress is comprised of a first edge region that has 8 layers and a middle region that has 10 layers. However, it can also be provided that the compress has a homogenous layer structure of either 10 layers or 12 layers or 16 layers.

Here, in particular, the outer edges C of the finished folded compress form folded edges that are parallel to the first folded edges, and the outer edges D of the finished folded compress form folded edges that are parallel to the second folded edges, wherein, as is further also, the first folded edge connects the two outer material sections forming the contact surfaces of the compress, and the second outer edge is located perpendicular thereto. In addition, it has been found that a compress according to the present disclosure, the outer edges of which are formed exclusively by folded edges, can be particularly securely used. This arrangement of the layers has the advantage that each additional layer of the compress is arranged between the two outer layers, and in this way, a compress can be provided that can be easily grasped by the user. So even if the compresses according to the present disclosure are arranged in a stack, a compress can be grasped without accidentally grasping an additional layer of an adjacent compress.

In another form of the present disclosure, it can also be provided that the compress has two additional cut and/or folded edges as second hems. In particular, these second hems are located parallel to the second folded edge. Additionally, the second hems are preferably located perpendicular to the first hems. In this way, it can be ensured that all cut edges are arranged on the inside of the compress.

According to the present disclosure, the folded edges that form the first hems do not form outer edges of the compress. The first hems, therefore, are always located between two outer edges, which, in particular, are located parallel to each other. According to yet another form of the present disclosure, it is provided that the compress comprises first hems which, at any point, have a distance from a first outer edge of at least about 25% and no more than about 75% of the amount of the length of a second outer edge that is located perpendicular to the first outer edge. In another form, these compresses comprises first hems which, at any point, have a distance from a first outer edge of at least about 40% and no more than about 60% of the length of a second outer edge that is located perpendicular to the first outer edge. In still another form, these compresses comprise first hems which, at any point, have a distance from a first outer edge of at least about 45% and no more than about 55% of the amount of the length of a second outer edge that is located perpendicular to the first outer edge.

In this way, another form of the present disclosure provides a compress that is folded in such a way that the folded-in cut edges do not, as in known ES compresses, rest one above the other, but rather are located in the middle of the compress next to each other. This produces the additional benefit that two additional layers are present where they can be useful.

In addition, a compress is provided that is flatter and therefore takes up less storage space. In an additional comparison to a known 8-layer ES compress, an 8-layer compress according to the present disclosure, at the thickest point thereof, comprises only 10 layers, while the known ES compress has 16 layers. Due to the folding according to the present disclosure, a compress can be provided that is much more easily stackable.

The flat web textile material for manufacturing a compress according to the present disclosure can be any flat web textile material that is different from non-woven flat web materials, which are referred to as non-wovens or non-woven fabrics. The present disclosure does not relate to any non-woven compresses. According to the present disclosure, woven or knitted fabrics can be used as flat web textile materials. Woven fabrics, and in particular woven fabrics with a plain weave, are particularly preferred.

In addition, the flat web textile materials can be manufactured from yarn or fiber material that comprises of fibers or filaments of natural origin and/or synthetic fibers. Fibers of natural original, which an inventive compress according to the present disclosure comprises, include particularly cotton, hemp, flax, or linen. If the flat web material contains yarn or fiber material that includes synthetic fibers, these can be fibers or filaments of viscose, polyester, cellulose acetate, carboxymethyl cellulose, and hydroxyethyl cellulose. In yet another form, the flat web textile material comprises a yarn or fiber material made of cotton and/or viscose that meets the standards of DIN EN 14079.

These materials, particularly gauze, in contrast to known non-textile or non-woven flat web materials such as non-woven or non-woven fabrics cannot be processed in a continuous, stageless process. For this reason, compresses according to the present disclosure must be manufactured from a discrete material section.

Another form of a medical compress according to the present disclosure is a gauze compress. This gauze compress comprises at least 8 layers of gauze in accordance with DIN EN 14079, wherein each layer is connected via at least one folded edge to an additional layer, and wherein at least one first folded edge and a second folded edge are located perpendicular to each other. The gauze compress is folded in such a way that the compress comprises at least two folded edges as first hems, wherein each of these first hems connects directly adjacent layers. In particular, the medical compress comprises two folded edges as first hems and two cut edges as second hems. In addition, this gauze compress can exhibit all of the additional characteristics of the previously described type, individually or in combination.

According to a continuative idea of the present disclosure, a stack of compresses comprising a plurality of the medical compresses is also provided. This stack comprises a plurality of compresses of the previously described type. In particular, this stack can be comprised of a plurality of identical compresses, wherein each compress exhibits individual characteristics or combinations of characteristics of the previously described compresses.

Accordingly, in particular, the present disclosure also provides a stack of compresses comprised of a plurality of medical compresses including at least 8 layers of a flat web textile material, in particular a plurality of gauze compresses including 8 layers according to DIN EN 14079. Each of these at least 8 layers of each individual compress is connected via at least one folded edge to an additional layer of this compress, wherein at least one first and one second folded edge are located perpendicular to each other. Each of these compresses is folded in such a way that it comprises at least two folded edges as first hems, wherein each of these first hems connects directly adjacent layers or partial sections of the directly adjacent layers.

By arranging the folded ends as hems, a compress stack can be provided which, compared to the compresses available in the market, is more stable and takes up less space. In this way, in particular packaging material can be saved. When, for example, the ES compresses currently available in the market are stacked, the package containing a stack of 100 compresses measures 155 mm in height (outside dimensions of the package). However, when the compresses according to the present disclosure are stacked (100 pieces) under the same conditions, the outside dimension is 130 mm (under the same measuring conditions). Thus, in particular packaging material and storage space can be saved.

In addition, a stack of compresses according to the present disclosure advantageously comprises rectangular or square contact surfaces, with each compress having two mutually opposing outer edges C having an edge length c, and two mutually opposing outer edges D having an edge length d, and with the edge length c being greater than or equal to the edge length d. In particular, this stack comprises a plurality of compresses having square contact surfaces.

In another form of the present disclosure, a stack of compresses according to the present disclosure comprises a plurality of compresses, the first hems of which, at each of the points thereof, have a distance from a first outer edge of at least about 25% and no more than about 75% of the amount of the length of a second outer edge that is located perpendicular to the first outer edge. In another form, these compresses comprise first hems which, at any point, have a distance from a first outer edge of at least about 40% and no more than about 60% of the amount of the length of a second outer edge that is located perpendicular to the first outer edge. In yet another form, these compresses have first hems, which, at any point, have a distance from a first outer edge of at least about 45% and no more than about 55% of the amount of the length of a second outer edge that is located perpendicular to the first outer edge.

These compresses are stacked one over the other in such a way that in each case, a first contact surface of a first compress is superimposed congruently with a first contact surface of a second or further compress. Here it can also be preferred that the first folded edges of a compress, which connects the two outer layers of the flat web textile material forming the contact layers of the compress to each other, are superimposed congruently with each other.

In continuation of the present disclosure, a method for manufacturing a medical compress having at least 8 layers of a flat web textile material is provided, wherein each layer is connected via at least one folded edge to an additional layer and at least one first and one second folded edge are disposed perpendicular to each other. In particular, a method for manufacturing a compress of the previously described type is to be disclosed. The method comprises the following steps:

a) Providing a rectangular material section of the flat web material having two mutually opposing first cut edges A and two mutually opposing second cut edges B, wherein the edge length a of the cut edge A is greater than or equal to the edge length b of the cut edge B,

b) Folding in the second cut edges B of the rectangular material section to form two first folded edges

c) Folding in the folded edges formed in b) to form two first hems of the compress,

d) Additional folding in of folded edges to form additional folded edges or hems of the compress.

In particular, the method includes the step a) Providing a rectangular material section of the flat web material having two mutually opposing first cut edges A and two mutually opposing second cut edges B, wherein the edge length a of the cut edge A is greater than the edge length b of the cut edge B.

Within the step d), it is furthermore preferred to fold in the first cut edges A of the rectangular material section to form two hems, wherein in particular step d) takes place before step c). In addition, as an additional step e), further folding in of folded edges may be carried out to form additional folded edges or hems.

Furthermore, it is preferred if in the step b) no hems are formed by the cut edges B. However, it can also be provided that in step b) two folded edges and two hems that are different from these folded edges can be formed, wherein the hems are formed by the cut edges B. It should be noted at this juncture that the compresses according to the present disclosure can be manufactured both by machine and by hand.

The method according to the present disclosure is intended in particular to provide a method for manufacturing a medical compress that comprises at least 8 and no more than 16 layers of a flat web material. In one form, the method according to the present disclosure relates to a method for manufacturing a compress which, viewed in a cross-section, comprises 8-layer and 10 or 12-layer regions. This should be understood to mean that the compress, viewed in a cross-section, does not have a homogenous layer structure across the entire region of the transverse or longitudinal extension thereof, but rather that the compress has 10 or 12 layers in a first partial region and 8 layers in at least one additional partial region. However, it can also be provided that the compress has a homogenous layer structure of either 10 layers or 12 layers or 16 layers.

In particular, a method for manufacturing a compress having square contact surfaces F with the surface measure d2 is to be provided, wherein d is the edge length of an outer edge D of the compress. In this method, a rectangular material section having the cut edges A and B is preferably used as the starting material, wherein this material section also preferably has an edge length a with a=4d+2e of the cut edges A and an edge length b with b=d+2e′ of the cut edges B, where d is the edge length of the finished folded compress, e the edge length of a partial section of the cut edge A with e<½d, and e′ the edge length of a partial section of the cut edge B with e′<½d. In this way, without major cuttings or waste, a compress comprising at least 8 layers and no more than 10 layers can be produced without major cuttings or waste, said compress providing a particularly even distribution of material across the contact surface.

If, on the other hand, the same material section is used and the folded edges produced in step b) are not formed as hems, a compress that is limited to 8 layers is given a smaller contact surface. Expressed in different terms, this circumstance means that a larger material section must be provided in order to manufacture a compress having a defined contact surface.

In another form, in this method therefore step b) is carried out before step d). In particular, in this method the cut edges B of the material section provided are folded in during step b, said cut edges having an edge length b with b=d+2e′. In this way, in contrast to a method in which the cut edges A are folded in during b), said cut edges having an edge length of a=4d+2e, a compress is obtained that has a larger contact surface with the same material usage.

It should be stressed at this juncture that the characteristics listed here for the preferred or alternative embodiments of the inventions are not limited to the individual preferences or alternatives. In addition, the combination of the various forms, or the combination of the individual characteristics of the alternative forms, is also considered part of the present disclosure. By the same token, the present disclosure shall not be understood as limited by the following description of the drawings.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1: A material section for manufacturing a compress according to the present disclosure viewed from above;

FIG. 2a: An intermediate product for manufacturing a compress according to the present disclosure viewed from above;

FIGS. 2b, 2c, 2d: An intermediate product according to FIG. 2a in various cross-sections;

FIG. 3a: An intermediate product for manufacturing a compress according to the present disclosure viewed from above;

FIGS. 3b, 3c, 3d: An intermediate product according to FIG. 3a in various cross-sections;

FIG. 4a: A compress according to the present disclosure viewed from above;

FIGS. 4b, 4c, 4d, 4e: The compress according to FIG. 4a in different cross-sections;

FIG. 5: A further compress according to the present disclosure viewed from above.

The drawings described herein are for illustration purposes only and are not intended to limit the scope of the present disclosure in any way.

### DETAILED DESCRIPTION

The following description is merely exemplary in nature and is not intended to limit the present disclosure, application, or uses.

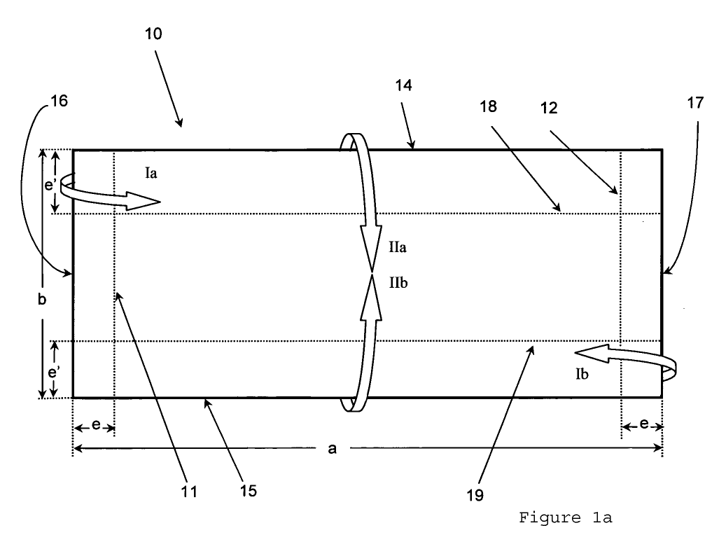

FIG. 1 shows a rectangular material section (10) of gauze material according to DIN EN 14079 for producing a compress according to the present disclosure having square contact surfaces. This material section comprises two mutually opposing first cut edges A (14, 15) having the edge length a=230.0 mm. The material section also comprises two mutually opposing second cut edges B (16, 17) having the edge length b=99.0 mm.

The following is a description of a method for manufacturing a compress having at least 8 layers based on the drawings. In a first step, the described rectangular material section (10) is provided. In a second step, the cut edges B (16, 17) are folded over onto an upper side of the material section in the direction of the arrows Ia and Ib along the fold lines I (11, 12) in order to form first folded edges G (26) and G′ (27). By folding over a cut edge B (16, 17), two subunits of the material section are laid one over the other such that the folded-over subunit and the remaining subunit form, in each case, a separate layer, whereby the layers formed are directly adjacent and connected the folded edges G (26) and G′ (27) that are formed. The distances e of the fold lines I to the closer, parallel cut edge B in each case are e=14.5 mm. Then, in a third step, the cut edges (14, 15) are folded over onto an upper side of the material section in the direction of the arrows IIa and IIb along the fold lines II (18, 19) to form second folded edges H (24) and H′ (25) I. The distances e′ of the fold lines II (18, 19) to the respectively closer, parallel cut edge A in each case is 24.5 mm. The second folded edges H (24) and H′ (25), as well as the hems (28a, 29a; 28b, 29b, 28b′, 29b′) formed by partial regions of the cut edges A, are formed by folding over the cut edges A (14, 15). Additional partial regions of these hems form the second hem of the finished compress.

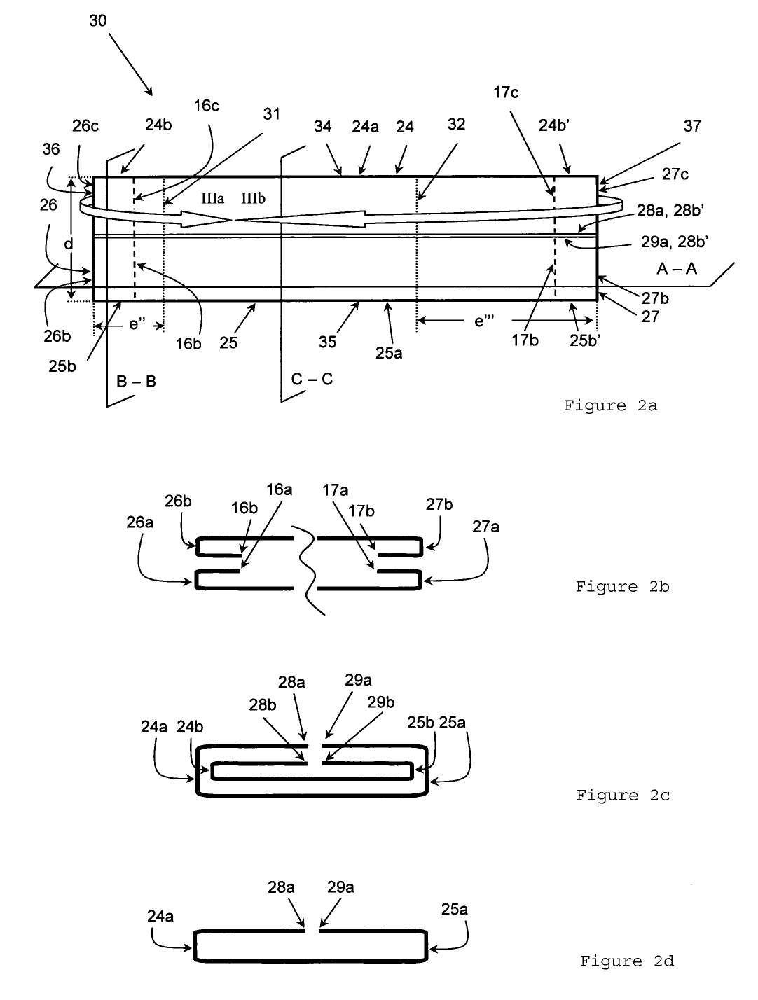

FIG. 2a shows the intermediate product A (30) comprising the outer edges bb (36) and bb′ (37) located parallel to the cut edges B, and the outer edges aa (34) and aa′ (35) located parallel to the cut edges A obtained after the third step. Here, the outer edge aa (34) is formed by a partial section of the second folded edge H (24a) and the outer edge aa′ (35) by a partial section of the second folded edge H′ (25a), (cf. FIG. 2c—Cross-section of an intermediate product A along the cut line B-B; as well as FIG. 2d—Cross-section of the intermediate product A along the cut line C-c). The additional partial sections of the folded edges H and H′ (24b, 25b, 24b′, 25b′), which are enclosed by the partial sections that form the outer edges, do not form outer edges of the intermediate product. The outer edge bb (36) is formed by superimposed partial sections of the first folded edge G (26a, 26b, 26c), and the outer edge bb′ (37) is formed by superimposed partial sections of the first folded edge G′ (27a, 27b, 27c) (cf. also FIG. 2b—Cross-section of the intermediate product A along the cut line A-A). By folding over the cut edges A (14, 15), hems are formed by the cut edges A, the hems being spaced 1.0 mm from each other in each case. All partial regions of the cut edges B (16a, 16b, 16c, 17a, 17b, 17c) are covered by material regions disposed on top (in the figures, the edges that are covered by material layers disposed on top are shown by dotted lines).

The intermediate product A (30) is further processed in a further step. For this purpose, the outer edges bb (36) and bb′ (37) that have been formed in the intermediate product A are folded over onto an upper side of the material section in the direction of the arrows IIIa and IIIb along the fold lines III (31, 32) in order to form the first hems (38a, 39a). The distance e″ of the first fold line III (31) from the closest, parallel outer edge aa (36) is e″=25.0 mm. The distance e′″ of the second fold line III (32) from the closest, parallel outer edge aa′ (37) is e′″=75.0 mm. By folding over the outer edges bb (36) and bb′ (37), additional folded edges I (41), I′ (42), I″ (43) and I′″ (44) as well as the first hems (38a, 39a) and additional hems (38b, 39b, 38c, 39c) of a level which is arranged further below of the finished compress are formed. Each formed hem (38a, 39a, 38b, 39b, 38c, 39c) is formed by partial sections of the folded edges G (26) and G′ (27), so that the hems connect directly adjacent layers. No further layers are inserted into or between the connected layers.

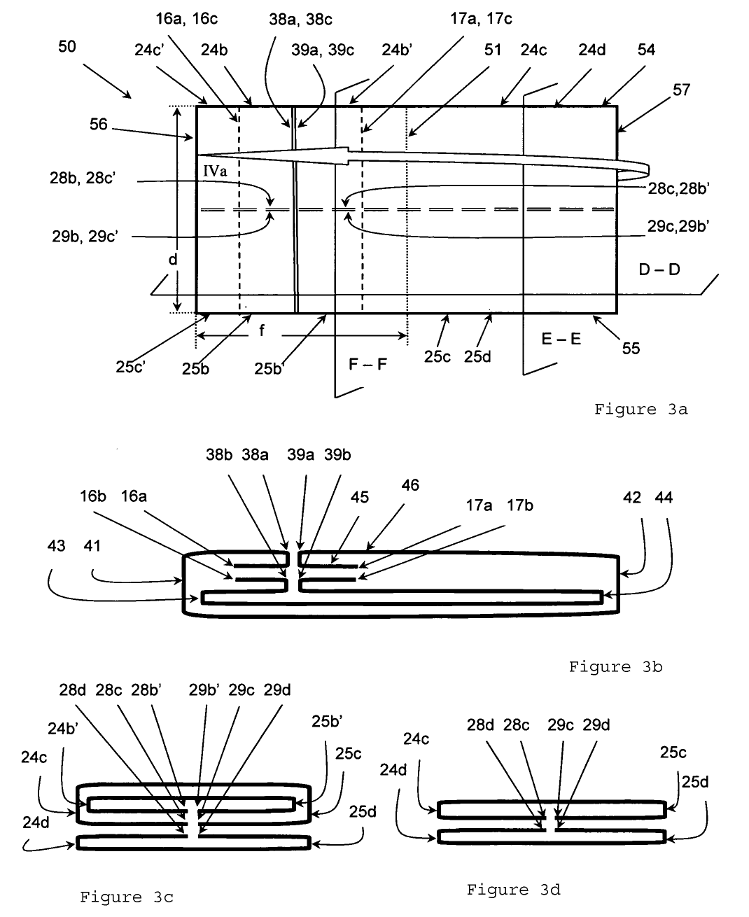

FIG. 3a shows the intermediate product B (50) obtained after the fourth step comprising the outer edges bbb (56) and bbb′ (57) parallel to the cut edges B, as well as the outer edges aaa (54) and aaa′ (55) parallel to the cut edges A. The outer edge aaa (54) is formed by partial sections (24c, 24d, 24c′) of the second folded edge H (24) that are located one above the other and, analogously, the outer edge aaa′ (35) is formed by partial sections (25c, 25d, 25c′) of the second folded edge H′ (25) that are located one above the other (cf. also FIG. 3c—Cross-section of the intermediate product B along the cut line F-F; as well as FIG. 3d—Cross-section of the intermediate product A along the cut line E-E). The additional partial sections of the folded edges H and H′ (24b, 25b, 24b′, 25b′) that are enclosed by the partial sections do not form outer edges of the intermediate product B. The outer edge bbb (56) is formed by the additional folded edge I (41) and, analogously, the outer edge aaa′ (57) is formed by the additional folded edge I′ (42) (cf. FIG. 3b—Cross-section of the intermediate product B along the cut line D-D). The additional folded edges of the inner layers I′ (43) and I″ (44) do not form outer edges of the intermediate product B (50). The first hems are formed by partial sections of the folded edges G and G′ (38a, 39a). In an additional plane, additional hems (38b, 39b, 38c, 39c) are formed by additional partial sections of the folded edges G and G′. The first hems (38a, 39a) are located directly adjacent to each other, the distance between them being 1.0 mm. The distance corresponds to a distance of 2% relative to the edge length d of an outer edge d of the finished compress. Both the cut edges B, or the partial sections (16a, 16b, 17a, 17b, 16c, 17c) thereof, and the hems (28b, 29b, 28b′, 29b′, 28c, 29c, 28c′, 29c′, 28d, 29d) that are formed by partial sections of the cut edges A are covered by material layers located on top. In this way, there are no exposed cut edges in this intermediate product B (50).

In a final step, the intermediate product B (50) is finished. For this purpose, the outer edge bbb′ (57) of the intermediate product B (50), which was formed in a previous step by the folded edge I′ (42), is folded over in the direction of the arrow IVa along the fold line IV (51), so that the folded-over outer edge bbb′ (57) is placed onto the outer edge bbb (56) of the intermediate product B (50) formed by the folded edge I. The distance between the fold line IV (51) and the outer edge bbb (56) corresponds to f=50.5. As a result of this step, a folded edge J (47) that forms the first outer edge D′ (67) of the finished product and further folded edges J′, J″ and J′″ (48a, 48b and 48c) are formed.

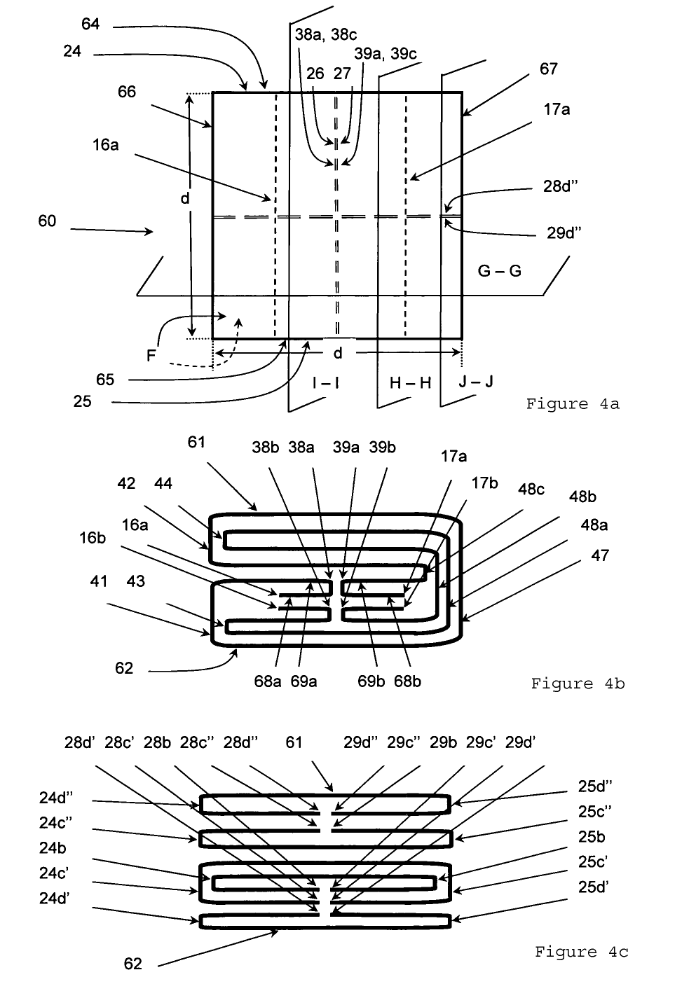

FIG. 4a shows the compress (60) produced in the previously described method. The compress, having the four outer edges D′, D″, D′″, and D″″ (64, 65, 66, and 67) having the same length and an edge length d=50.0 mm, comprises square contact surfaces f having the surface measure d2=25.0 cm2, two folded edges (38a, 39a) configured as first hems, and two partial regions of the cut edges A as second hems (28d″, 29d″). All partial regions of the cut edges B and all partial regions of the cut edges A are covered by additional material layers. In FIG. 4a, only the upper partial regions of the cut edge B (16a, 17a) and the partial regions of the cut edge A (28d″, 29d″) that form the second hems are illustrated. The first hems (38a, 39a), which are formed by a partial region of the folded edge G (26) and a partial region of the folded edge G′ (27), directly about each other, the distance between them amounting to 1.0 mm. The distance corresponds to a distance of about 2% relative to the edge length d of the outer edge D′ (all edges have the same length) of the finished compress. The second hems (28d″, 29d″) are spaced at a distance of 1.0 mm, the distance corresponding to about 2% relative to the edge length d of an outer edge D′. The first hems (38a, 39a) are located approximately in the middle of the compress and, at any point, have a distance to the parallel first outer edge D′ (67) off about 49%, or about 51%, of the amount of the length of a second outer edge (64), which is vertical to the first outer edge. In addition, the first hems (38a, 39a) are disposed perpendicular to the second hems (28d″, 29d″).

The following FIGS. 4b, 4c, 4d, and 4e are intended to clarify the layered structure of the compress (60), in FIG. 4b, the cross-section according to the cut line G-G being shown, in FIG. 4c the cross-section according to the cut line I-I, in FIG. 4d the cross-section according to cut line H-H, and in FIG. 4e the cross-section according to the cut line J-J. In order to clearly illustrate the layered structure of the compress (60), the individual layers and nested folded edges—as in all cross-section drawings—are shown in an exploded view, or at a distance from each other. In particular, it is shown that the two outer layers (61, 62), which form the contact surfaces F, are connected to each other by the folded edge J (47), which forms the first outer edge D′ (67) of the compress. Each additional layer is surrounded by these two outer layers, so that all additional layers are located between the outer layers (61, 62).

Furthermore, it is shown that the second outer edge D″ (64) of the compress is formed by partial sections of the folded edge H (24c′″, 24d′, 24c″, 24d″, 24c′) located one over the other, the third outer edge D′″ (45) by partial sections of the folded edge H′ (25c′″, 25d′, 25c″, 25d″, 25c′) located one over the other, and the fourth outer edge D″″ (66) by the folded edges I (41) and I′ (42) located one over the other. The folded edges (43, 44, 48a, 48b, 48c) located inside and the other partial sections of the folded edges H (24b, 24b′) and H′ (25b, 25b′) do not form any outer edges of the compress. As a result, the compress comprises only outer edges that are formed by folded edges. Furthermore the compress comprises congruent first hems (38a, 39a) and additional hems (38b, 39b) formed by folded edges. These hems are formed by the folded edges G (26) and G′ (27). Each of these first hems (38a, 39a) connects a first layer directly to a second layer, wherein the first layer is formed by a first partial section (68a) and a second partial section (69b) and the second layer is formed by a first partial section (69a) and a second partial section (69b) (cf. FIG. 4b). Located perpendicular thereto are the congruent second hems (28d′″, 29d″) formed by the cut edges and additional hems (28′″, 29c′″; 28c″, 29c″, 28c′, 29c′; 28d′, 29d′; 28b, 29b, 28b′, 29b′) formed by the cut edges A.

Each layer of this compress is connected by at least one folded edge to an additional layer of the compress, wherein the compress, viewed in the cross-section, comprises 8-layer and 10-layer regions. As an 8-layer compress, the compress has a middle partial region that has 10 layers. The two additional layers are obtained by folding over the cut edges B (16, 17) (cf. FIG. 4b). In the edge regions, the compress has 8 layers (cf. FIG. 4d). This compress, as an 8-layer compress, also has no exposed cut edges (cf. FIG. 3a) after being unfolded once. Consequently, this compress can be used either as a 4-layer or an 8-layer compress. In the middle region (10 layers) the compress (60) has a thickness of 1.24 mm, and in the edge region (8 layers), it has a thickness of 1.12 mm, measured in each case with a test pressure of 2 g/cm² (See below). In this way, a plurality of these compresses can be easily stacked, because they have a uniform distribution of material across all regions.

A comparison of stack heights will be made below. If, for example, the ES compresses available in the market (ES compresses 5×5 cm—Paul Hartmann AG) are stacked, the package comprising a stack of 100 compresses is 155 mm high (outside dimension of the package). In contrast, if 100 units of the compress (60) according to the present disclosure are stacked and packaged under the same conditions with the same packaging material, the outside dimension is 130 mm (under the same measuring conditions). This corresponds to a reduction in the stack height of approximately 16%.

For a partial stack of 5 compresses, or for each individual compress, the following values are obtained, using a test pressure of 2 g/cm2. Each compress is folded identically, and the compresses in the stack are stacked congruently.

| | | Thickness | Thickness |
|---|---|---|---|
| | | (5 pieces)/mm | (1 piece)/mm |
| ES compress | Outer edge | 8.10 | 1.62 |
| (Paul Hartmann | (16 layers) | | |
| AG) | | | |
| | Parallel opposite | 5.65 | 1.13 |
| | edge | | |
| | (8 layers) | | |
| Compress (60) | Edge region | 5.60 | 1.12 |
| according to | (8 layers) | | |
| present | | | |
| disclosure | | | |
| | Middle region | 6.20 | 1.24 |
| | (10 layers) | | |

By arranging the folded edges as hems, a compress stack can be prepared which, compared to the compresses currently available in the market, is more stable and takes up less space.

In the following table, examples of square compresses are provided, which according to the method described above are formed by a rectangular material section having two mutually opposing parallel cut edges A and two mutually opposing parallel cut edges B. The edge length a of the cut edges A is greater than the edge length b of the cut edges B of the material section provided. The compresses compared, in a finished folded state, have the outer edge length d that is specified in each case. The table is intended to point out the material savings that are possible, using the same material, compared to known ES compresses—Paul Hartmann AG (1st fold), wherein the first folded edges of the ES compresses used as a comparison are formed parallel to the cut edges A.

| | Surface of the material section used F_{M} = a · b | | | | |
|---|---|---|---|---|---|
| | Compress | | | Compress | Compress |
| | 1 | | | 2 | 3 |
| | d = 5 cm | | | d = 7.5 cm | d = 10 cm |
| | e = 1.0 cm | e = 1.5 cm | e = 2.0 cm | e = 1.5 cm | e = 1.5 cm |
| 1^{st} fold | 240 cm² | 260 cm² | 280 cm² | 540 cm² | 920 cm² |
| ES | | | | | |
| compress | | | | | |
| F_{M} = 4d · | | | | | |
| (2d + 2e) | | | | | |
| 2^{nd} fold | 220 cm² | 230 cm² | 240 cm² | 495 cm² | 860 cm² |
| (compress | | | | | |
| acc. to | | | | | |
| present | | | | | |
| disclosure) | | | | | |
| F_{M}= (4d + | | | | | |
| 2e) · 2d | | | | | |
| Material | 20 cm² | 30 cm² | 40 cm² | 45 cm² | 60 cm² |
| savings | (9.1%) | (11.5%) | (14.3%) | (8.3%) | (6.5%) |

Thus, the material savings for a compress according to the present disclosure having square contact surfaces and an edge length d with d=5 cm (compress 1), and having the same width e of the section folded over in the first step with e=1.5 cm, amount to approximately 11.5% compared to established ES compresses. Here, both the first and the second hems have a distance of 1.0 mm. The material savings can thus be achieved by precise folding in a novel manner, the hems according to the present disclosure being realized through the folding.

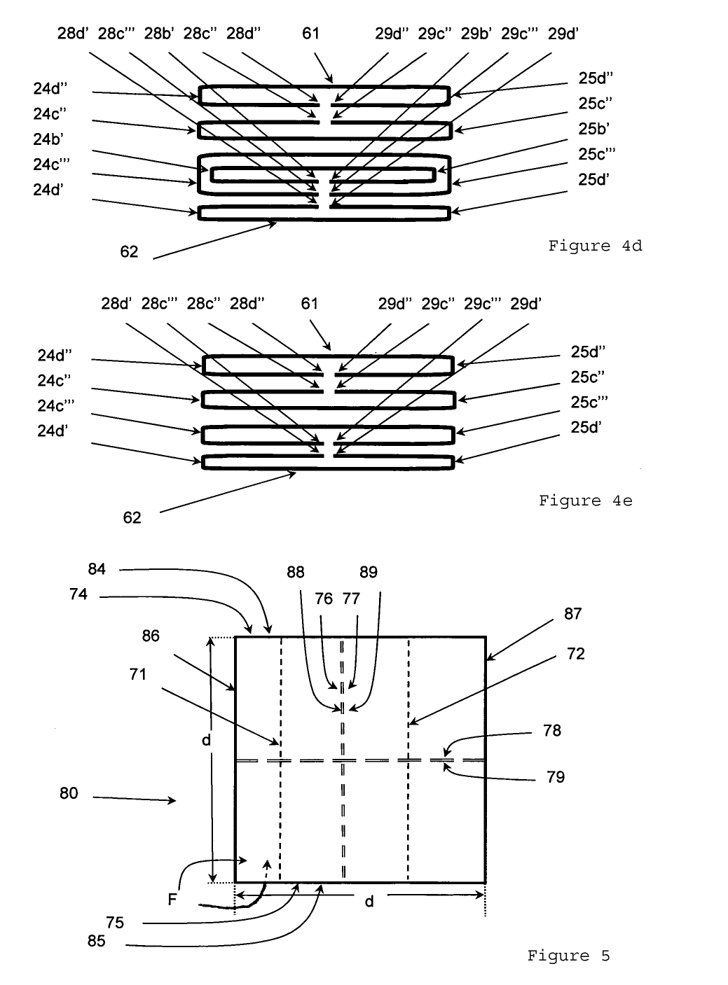

FIG. 5 shows a further example of an 8-layer gauze compress that has square contact surfaces. The edge length d of the outer edge D of the compress (80) is d=75.0 mm. The compress is manufactured according to the previously described method, wherein the following dimensions were observed: a=330.0 mm, b=149.0 mm, e=14.5 mm, e′=37.0 mm, e″=30.0 mm, e′″=120.0 mm and f=75.5 mm. This compress thus has four outer edges D′, D″, D′″, D″″ (84, 85, 86, and 87) of equal length, covered cut edges B (71, 72), folded edges (88, 89) configured as first hems, and cut edges A (78, 79) configured as second hems. The distance between the second hems (78, 79) formed is 1.00 mm (1.3% relative to the edge length d of the outer edge D′ (87) of the compress). The distance between the first hems (88, 89) formed is 1.0 mm (1.3% relative to the edge length d of the outer edge D′ (87) of the compress). The distance between the first hems (88, 89) and the first outer edge D′ (87) located parallel to the hems is about 60%, or about 58.6%, relative to the edge length of a second outer edge D″ (84) of the compress located perpendicular to the first. If the distance is considered relative to the second outer edge D′″ (86) located parallel to the first hems, the distance is about 40%, or about 41.3%. In any case, the hems are located in a middle region of the contact surfaces of the compresses that extends parallel to a first outer edge in the direction of the second parallel outer edge at a distance of about 25 to about 75% of the edge length of the edge which in turn extends perpendicular to the first or second outer edge.

The medical compresses shown here can be used particularly for emergency treatment as well as during surgical procedures. They are characterized by particular security in use and by a particularly even distribution of material.

It should be noted that the disclosure is not limited to the various forms described and illustrated as examples. A large variety of modifications have been described and more are part of the knowledge of the person skilled in the art. These and further modifications as well as any replacement by technical equivalents may be added to the description and figures, without leaving the scope of the protection of the disclosure and of the present patent.

What is claimed is:

1. A medical compress comprising at least 8 layers of a flat web textile material, each layer being connected by at least one folded edge to an additional layer, and at least one first folded edge and one second folded edge being located perpendicular to each other, characterized in that the compress is folded in such a way that the compress comprises at least two folded edges as first hems, wherein each of these first hems connects directly at least one of adjacent layers and partial sections of the directly adjacent layers.

2. The medical compress according to claim 1, characterized in that the compress is folded in such a way that the outer layers that form the contact surfaces of the compress are in each instance formed completely of a coherent region of the flat web material.

3. The medical compress according to claim 1, characterized in that the first hems, at any point, have a distance to a parallel first outer edge of least about 25% and no more than about 75% of the amount of the length of a second outer edge located perpendicular to the first outer edge.

4. The medical compress according to claim 1, characterized in that the compress comprises at least one of two additional cut and folded edges as second hems.

5. The medical compress according to claim 1, characterized in that the first hems are located perpendicular to the second hems.

6. The medical compress according to claim 1, characterized in that each outer edge of the compress is formed exclusively by folded edges.

7. The medical compress according to claim 1, characterized in that the compress, viewed in a cross-section, comprises 8-layer and 10-layer regions.

8. The medical compress according to claim 1, characterized in that the compress has 10 layers.

9. A stack of compresses, comprising a plurality of compresses according to claim 1.

10. A method for manufacturing medical compress comprising at least 8 layers of a flat web textile material, each layer of the compress being connected by at least one folded edge to a further layer, and at least one folded edge and one second folded edge being located perpendicular to each other, in particular for manufacturing a compress according to at least one of the preceding claims, characterized in that the method comprises the following steps:
a) Providing a rectangular material section of the flat web material having two mutually opposed first cut edges A and two mutually opposed second cut edges B, wherein the edge length a of the cut edge A is greater than or equal to the edge length b of the cut edge B;
b) Folding in the second cut edges B of the rectangular material section (10) in order to form two first folded edges;
c) Folding in the folded edges formed under b) in order to form two first hems of the compress, and;
d) Additional folding in of the cut or folded edges to form additional folded edges or hems of the compress.

11. The method according to claim 10, characterized in that within step d), a folding in of the first cut edges A of the rectangular material section is carried out in order to form two second hems, wherein, as an additional step:
e) Additional folding-in of folded edges can be performed to form additional folded edges of hems.
